(19) United States
(12) Patent Application Publication
(10) Pub. No.: US 2022/0087871 A1
(43) Pub. Date: Mar. 24, 2022

(54) Abdominal Negative Pressure Therapy Dressing With Remote Wound Sensing Capability

(71) Applicant: KCI Licensing, Inc., San Antonio, TX (US)

(72) Inventors: Justin Alexander LONG, Lago Vista, TX (US); Christopher Brian LOCKE, Bournemouth (GB); Benjamin Andrew PRATT, Poole (GB)

(21) Appl. No.: 17/424,436

(22) PCT Filed: Jan. 8, 2020
(86) PCT No.: PCT/US2020/012656

§ 371 Date: Jul. 20, 2021

Related U.S. Application Data
(60) Provisional application No. 62/800,252, filed on Feb. 1, 2019.

(51) Int. Cl. A61F 13/00; A61F 13/02; A61B 5/00; A61B 5/01

(52) U.S. Cl. CPC A61F 13/00068; A61F 13/0206; A61B 5/01; A61B 5/445; A61F 13/0216

(57) ABSTRACT

A system for applying negative-pressure therapy to an abdominal cavity. The system comprises: a tissue interface comprising a first contact layer and a second contact layer, each of the first contact layer and the second contact layer having perforations formed therein. The system also comprises a spacer layer disposed between the first contact layer and the second contact layer, the spacer layer configured to extend to different zones within the abdominal cavity. The system also includes a first sensor associated with the spacer layer and configured to acquire data associated with fluid at a first zone within the abdominal cavity and a first wireless transceiver associated with the first sensor and configured to transmit the data to an external therapy control device.

### RELATED APPLICATIONS

The present application claims priority to U.S. Provisional Patent Application No. 62/800,252, entitled “Abdominal Negative Pressure Therapy Dressing with Remote Wound Sensing Capability,” filed Feb. 1, 2019, which is incorporated herein by reference for all purposes.

### TECHNICAL FIELD

The invention set forth in the appended claims relates generally to tissue treatment systems and more particularly, but without limitation, to abdominal negative pressure therapy systems.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to therapy systems, components, and processes may benefit healthcare providers and patients.

### BRIEF SUMMARY

New and useful systems, apparatuses, and methods for remote monitoring in a negative-pressure therapy environment are set forth in the appended claims. Illustrative embodiments are also provided to enable a person skilled in the art to make and use the claimed subject matter.

Specialized abdominal treatment systems have proven to have great utility in the management of abdominal wounds. These specialized systems are designed to manifold pressure evenly across the surface of the abdomen while removing fluid, yet also substantially reduce or prevent granulation of the delicate intestinal tract exposed within the abdominal cavity. This therapy may be further enhanced by providing an ability to monitor pressure in distinct zones or areas within the abdomen. It would be advantageous to provide an ability to inform the user if one part of the abdominal cavity is stagnating or if a bowel is perforated and generating fluid in a particular zone or area within the abdomen.

Abdominal treatment systems may also have the capability to perform controlled fluid instillation. It is desirable to know if the fluid has been distributed evenly, if it is stagnated, if it is pooling to one area of the abdomen or the presence of potential sepsis. This information may be advantageous for judging the frequency or number of instillation cycles required to lavage the abdomen, or for determining an appropriate volume of fluid to achieve a full clean and flush of the abdominal cavity.

In some embodiments, a negative-pressure treatment system may be configured to determine the pressure at multiple discrete points in the abdominal cavity and to detect the presence and pH of liquid contained at various discrete points of the abdominal cavity in order to detect bowel perforations or intra-abdominal sepsis. The system may include a wireless transceiver in some examples, which may to a tablet device or smartphone. On-board sensors can provide closed-loop feedback and updates on the status of therapy provided and may also indicate if clinician intervention may be required. In some embodiments, the system can be used as an addition to a negative pressure therapy system in an acute setting that automatically or simply pairs to a smart phone or tablet device and that is capable of further interpreting and broadcasting telemetry or controlled feedback generated from its on-board sensors monitoring the status of the negative-pressure therapy so that the data may be communicated to the user, clinician, or compiled in a cloud environment.

More generally, a system for applying negative-pressure therapy to an abdominal cavity may comprise: i) a tissue interface comprising a first contact layer and a second contact layer, each of the first contact layer and the second contact layer having perforations formed therein; ii) a spacer layer disposed between the first contact layer and the second contact layer, the spacer layer configured to extend to different zones within the abdominal cavity; iii) a first sensor associated with the spacer layer and configured to acquire data associated with fluid at a first zone within the abdominal cavity; and iv) a first wireless transceiver associated with the first sensor and configured to transmit the data to an external therapy control device. In some embodiments, the spacer layer may comprise an absorbent material.

In more particular examples, the first sensor can be configured to detect a presence of fluid in the first zone. In other embodiments, the first sensor can be configured to detect a pressure of fluid in the first zone. In still other embodiments, the first sensor can be configured to detect a temperature of fluid in the first zone. In yet other embodiments, the first sensor can be configured to detect a pH of fluid in the first appendage.

In further embodiments, the system may additionally comprise: a second sensor associated with the spacer layer and configured to acquire data associated with fluid at a second zone within the abdominal cavity; and a second wireless transceiver associated with the second sensor and configured to transmit to the external therapy control device at least one data parameter associated with the fluid in the second zone.

In some embodiments, the second sensor can be configured to detect a presence of fluid in the second zone. In other embodiments, the second sensor can be configured to detect at least one of: i) a pressure of fluid in the second zone; ii) a temperature of fluid in the second zone; and iii) a pH of fluid in the second zone.

In some embodiments, the external therapy control device can be configured to determine a difference between a first parameter value associated with fluid in the first zone and a corresponding first parameter value associated with fluid in the second zone.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a therapy system that can provide negative-pressure treatment in accordance with this specification.

FIG. 2 is a graph illustrating additional details of example pressure control modes that may be associated with some embodiments of the therapy system of FIG. 1.

FIG. 3 is a graph illustrating additional details that may be associated with another example pressure control mode in some embodiments of the therapy system of FIG. 1.

FIG. 4 is an assembly diagram illustrating additional details that may be associated with an exemplary tissue interface of the dressing in FIG. 1.

FIG. 5 is a top view illustrating still more details that may be associated with the exemplary tissue interface of the dressing in FIG. 1.

FIG. 6 illustrates an exemplary pattern of perforations that may be associated with an alternate embodiment of the tissue interface in FIG. 4.

FIG. 7 is a perspective view of an exemplary tissue interface applied to a tissue site that comprises an abdominal cavity.

FIG. 8 is a schematic diagram of the wireless capable sensors associated with the exemplary tissue interfaces.

FIG. 9 is a network topology diagram illustrating the operation of the wireless capable sensors in FIG. 8.

FIG. 10 is a flow diagram illustrating the operation of the wireless capable sensors in FIG. 8.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

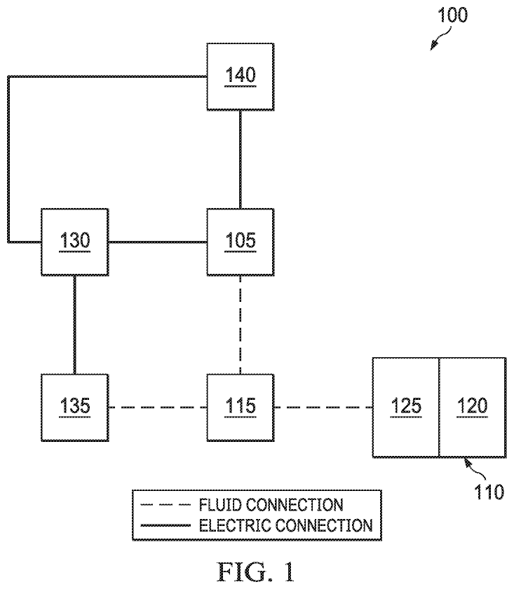

FIG. 1 is a simplified functional block diagram of an example embodiment of a therapy system 100 that can provide negative-pressure therapy to a tissue site in accordance with this specification.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 105, and one or more distribution components. A distribution component is preferably detachable and may be disposable, reusable, or recyclable. A dressing, such as a dressing 110, and a fluid container, such as a container 115, are examples of distribution components that may be associated with some examples of the therapy system 100. As illustrated in the example of FIG. 1, the dressing 110 may comprise or consist essentially of a tissue interface 120, a cover 125, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from Kinetic Concepts, Inc. of San Antonio, Tex.

The therapy system 100 may also include a regulator or controller, such as a controller 130. Additionally, the therapy system 100 may include sensors to measure operating parameters and provide feedback signals to the controller 130 indicative of the operating parameters. As illustrated in FIG. 1, for example, the therapy system 100 may include a first sensor 135 and a second sensor 140 coupled to the controller 130.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 130 and other components into a therapy unit.

In general, components of the therapy system 100 may be coupled directly or indirectly. For example, the negative-pressure source 105 may be directly coupled to the container 115 and may be indirectly coupled to the dressing 110 through the container 115. Coupling may include fluid, mechanical, thermal, electrical, or chemical coupling (such as a chemical bond), or some combination of coupling in some contexts. For example, the negative-pressure source 105 may be electrically coupled to the controller 130 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 105, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 115 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 130, may be a microprocessor or computer programmed to operate one or more components of the therapy system 100, such as the negative-pressure source 105. In some embodiments, for example, the controller 130 may be a microcontroller, which generally comprises an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 120, for example. The controller 130 is also preferably configured to receive one or more input signals, such as a feedback signal, and programmed to modify one or more operating parameters based on the input signals.

Sensors, such as the first sensor 135 and the second sensor 140, are generally known in the art as any apparatus operable to detect or measure a physical phenomenon or property, and generally provide a signal indicative of the phenomenon or property that is detected or measured. For example, the first sensor 135 and the second sensor 140 may be configured to measure one or more operating parameters of the therapy system 100. In some embodiments, the first sensor 135 may be a transducer configured to measure pressure in a pneumatic pathway and convert the measurement to a signal indicative of the pressure measured. In some embodiments, for example, the first sensor 135 may be a piezo-resistive strain gauge. The second sensor 140 may optionally measure operating parameters of the negative-pressure source 105, such as a voltage or current, in some embodiments. Preferably, the signals from the first sensor 135 and the second sensor 140 are suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal.

The tissue interface 120 can be generally adapted to partially or fully contact a tissue site. The tissue interface 120 may take many forms, and may have many sizes, shapes, or thicknesses, depending on a variety of factors, such as the type of treatment being implemented or the nature and size of a tissue site. For example, the size and shape of the tissue interface 120 may be adapted to the contours of deep and irregular shaped tissue sites. Any or all of the surfaces of the tissue interface 120 may have an uneven, coarse, or jagged profile.

In some embodiments, the tissue interface 120 may comprise or consist essentially of a manifold. A manifold in this context may comprise or consist essentially of a means for collecting or distributing fluid across the tissue interface 120 under pressure. For example, a manifold may be adapted to receive negative pressure from a source and distribute negative pressure through multiple apertures across the tissue interface 120, which may have the effect of collecting fluid from across a tissue site and drawing the fluid toward the source. In some embodiments, the fluid path may be reversed, or a secondary fluid path may be provided to facilitate delivering fluid, such as fluid from a source of instillation solution, across a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 120 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 120 may also vary according to needs of a prescribed therapy. For example, the tensile strength of foam may be increased for instillation of topical treatment solution. The 25% compression load deflection of the tissue interface 120 may be at least 0.35 pounds per square inch, and the 65% compression load deflection may be at least 0.43 pounds per square inch. In some embodiments, the tensile strength of the tissue interface 120 may be at least 10 pounds per square inch. The tissue interface 120 may have a tear strength of at least 2.5 pounds per inch. In some embodiments, the tissue interface may be foam comprised of polyols such as polyester or polyether, isocyanate such as toluene diisocyanate, and polymerization modifiers such as amines and tin compounds. In some examples, the tissue interface 120 may be reticulated polyurethane foam such as found in GRANUFOAM™ dressing or V.A.C. VERAFLO™ dressing, both available from Kinetic Concepts, Inc. of San Antonio, Tex.

The thickness of the tissue interface 120 may also vary according to needs of a prescribed therapy. For example, the thickness of the tissue interface may be decreased to reduce tension on peripheral tissue. The thickness of the tissue interface 120 can also affect the conformability of the tissue interface 120. In some embodiments, a thickness in a range of about 5 millimeters to 10 millimeters may be suitable.

The tissue interface 120 may be either hydrophobic or hydrophilic. In an example in which the tissue interface 120 may be hydrophilic, the tissue interface 120 may also wick fluid away from a tissue site, while continuing to distribute negative pressure to the tissue site. The wicking properties of the tissue interface 120 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Tex. Other hydrophilic foams may include those made from polyether. Other foams that may exhibit hydrophilic characteristics include hydrophobic foams that have been treated or coated to provide hydrophilicity.

In some embodiments, the tissue interface 120 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The tissue interface 120 may further serve as a scaffold for new cell-growth, or a scaffold material may be used in conjunction with the tissue interface 120 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source. The cover 125 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 125 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 125 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polyamide copolymers. Such materials are commercially available as, for example, Tegaderm® drape, commercially available from 3M Company, Minneapolis Minn.; polyurethane (PU) drape, commercially available from Avery Dennison Corporation, Pasadena, Calif.; polyether block polyamide copolymer (PEBAX), for example, from Arkema S.A., Colombes, France; and Inspire 2301 and Inspire 2327 polyurethane films, commercially available from Coveris Advanced Coatings, Wrexham, United Kingdom. In some embodiments, the cover 125 may comprise INSPIRE 2301 having an MVTR (upright cup technique) of 2600 g/m²/24 hours and a thickness of about 30 microns.

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

In operation, the tissue interface 120 may be placed within, over, on, or otherwise proximate to a tissue site. If the tissue site is a wound, for example, the tissue interface 120 may partially or completely fill the wound, or it may be placed over the wound. The cover 125 may be placed over the tissue interface 120 and sealed to an attachment surface near a tissue site. For example, the cover 125 may be sealed to undamaged epidermis peripheral to a tissue site. Thus, the dressing 110 can provide a sealed therapeutic environment proximate to a tissue site, substantially isolated from the external environment, and the negative-pressure source 105 can reduce pressure in the sealed therapeutic environment.

The fluid mechanics of using a negative-pressure source to reduce pressure in another component or location, such as within a sealed therapeutic environment, can be mathematically complex. However, the basic principles of fluid mechanics applicable to negative-pressure therapy and instillation are generally well-known to those skilled in the art, and the process of reducing pressure may be described illustratively herein as “delivering,” “distributing,” or “generating” negative pressure, for example.

In general, exudate and other fluid flow toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as a limiting convention.

Negative pressure applied across the tissue site through the tissue interface 120 in the sealed therapeutic environment can induce macro-strain and micro-strain in the tissue site. Negative pressure can also remove exudate and other fluid from a tissue site, which can be collected in container 115.

In some embodiments, the controller 130 may receive and process data from one or more sensors, such as the first sensor 135. The controller 130 may also control the operation of one or more components of the therapy system 100 to manage the pressure delivered to the tissue interface 120. In some embodiments, controller 130 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 130. The target pressure may vary from tissue site to tissue site based on the type of tissue forming a tissue site, the type of injury or wound (if any), the medical condition of the patient, and the preference of the attending physician. After selecting a desired target pressure, the controller 130 can operate the negative-pressure source 105 in one or more control modes based on the target pressure and may receive feedback from one or more sensors to maintain the target pressure at the tissue interface 120.

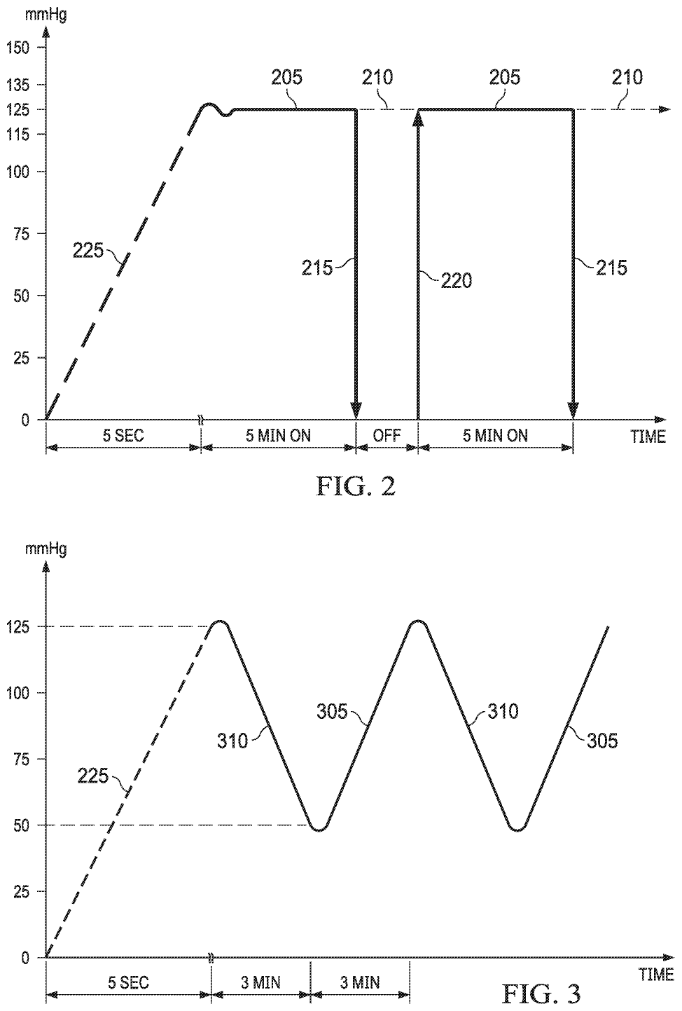

FIG. 2 is a graph illustrating additional details of an example control mode that may be associated with some embodiments of the controller 130. In some embodiments, the controller 130 may have a continuous pressure mode, in which the negative-pressure source 105 is operated to provide a constant target negative pressure, as indicated by line 205 and line 210, for the duration of treatment or until manually deactivated. Additionally, or alternatively, the controller may have an intermittent pressure mode, as illustrated in the example of FIG. 2. In FIG. 2, the x-axis represents time and the y-axis represents negative pressure generated by the negative-pressure source 105 over time. In the example of FIG. 2, the controller 130 can operate the negative-pressure source 105 to cycle between a target pressure and atmospheric pressure. For example, the target pressure may be set at a value of 135 mmHg, as indicated by line 205, for a specified period of time (e.g., 5 min), followed by a specified period of time (e.g., 2 min) of deactivation, as indicated by the gap between the solid lines 215 and 220. The cycle can be repeated by activating the negative-pressure source 105, as indicated by line 220, which can form a square wave pattern between the target pressure and atmospheric pressure.

In some example embodiments, the increase in negative-pressure from ambient pressure to the target pressure may not be instantaneous. For example, the negative-pressure source 105 and the dressing 110 may have an initial rise time, as indicated by the dashed line 225. The initial rise time may vary depending on the type of dressing and therapy equipment being used. For example, the initial rise time for one therapy system may be in a range of about 20-30 mmHg/second and in a range of about 5-10 mmHg/second for another therapy system. If the therapy system 100 is operating in an intermittent mode, the repeating rise time, as indicated by the solid line 220, may be a value substantially equal to the initial rise time as indicated by the dashed line 225.

FIG. 3 is a graph illustrating additional details that may be associated with another example pressure control mode in some embodiments of the therapy system 100. In FIG. 3, the x-axis represents time and the y-axis represents negative pressure generated by the negative-pressure source 105. The target pressure in the example of FIG. 3 can vary with time in a dynamic pressure mode. For example, the target pressure may vary in the form of a triangular waveform, varying between a negative pressure of 50 and 135 mmHg with a rise time 305 set at a rate of +25 mmHg/min. and a descent time 310 set at −25 mmHg/min. In other embodiments of the therapy system 100, the triangular waveform may vary between negative pressure of 25 and 135 mmHg with a rise time 305 set at a rate of +30 mmHg/min and a descent time 310 set at −30 mmHg/min.

In some embodiments, the controller 130 may control or determine a variable target pressure in a dynamic pressure mode, and the variable target pressure may vary between a maximum and minimum pressure value that may be set as an input prescribed by an operator as the range of desired negative pressure. The variable target pressure may also be processed and controlled by the controller 130, which can vary the target pressure according to a predetermined waveform, such as a triangular waveform, a sine waveform, or a saw-tooth waveform. In some embodiments, the waveform may be set by an operator as the predetermined or time-varying negative pressure desired for therapy.

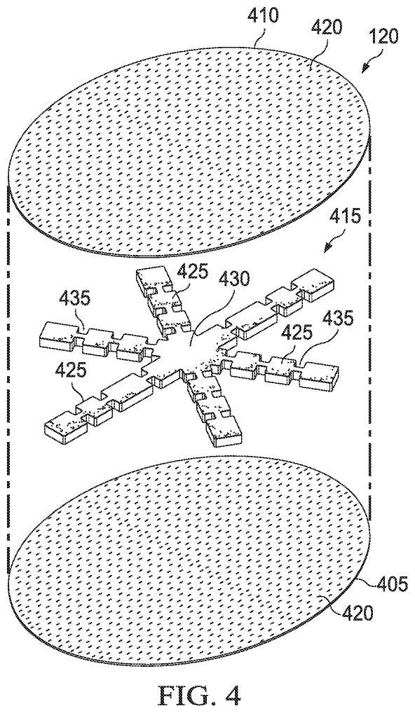

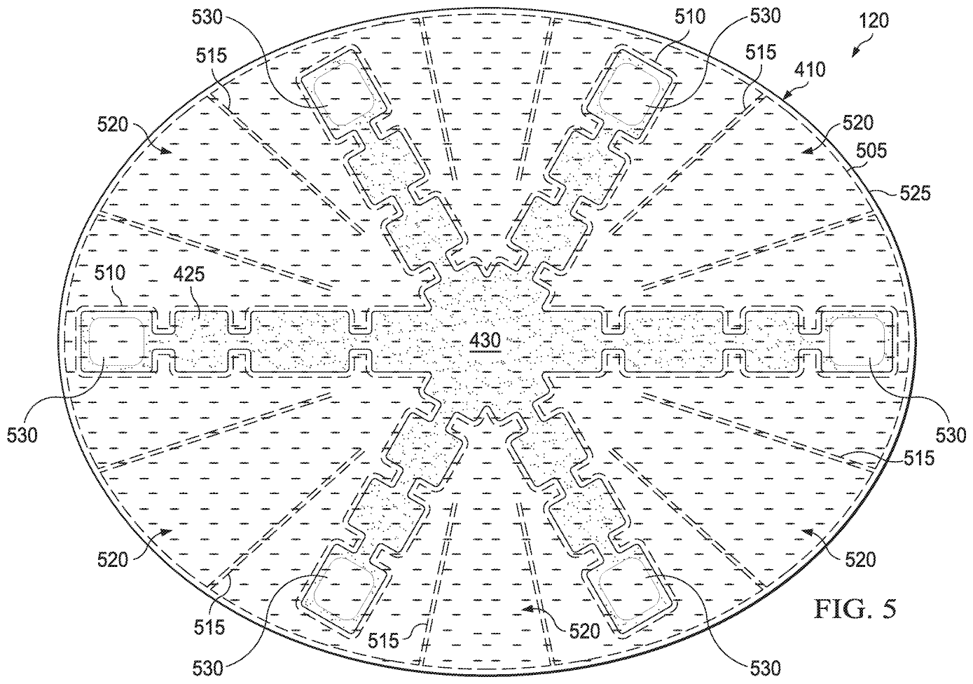

FIG. 4 is an assembly diagram of an example of the tissue interface 120, illustrating additional details that may be associated with some example embodiments having multiple layers. In the example embodiment of FIG. 4, the tissue interface 120 generally includes a first contact layer 405, a second contact layer 410, and a spacer layer 415. Each of the first contact layer 405, the second contact layer 410, and the spacer layer 415 may be a manifold. For example, as illustrated in FIG. 4, the first contact layer 405 and the second contact layer 410 may have fenestrations 420 suitable for distributing or collecting fluid across the tissue interface 120. The fenestrations 420 can have a variety of suitable shapes. For example, the fenestrations 420 may be circular or rectangular. In FIG. 4, the fenestrations 420 are slits. In some examples, the spacer layer 415 may be formed from a porous material, such as open-cell foam.

The first contact layer 405, the second contact layer 410, and the spacer layer 415 may also be sufficiently flexible to conform to a tissue site. For example, the first contact layer 405 and the second contact layer 410 may be a thin film of construction similar to the cover 125. A thickness of about 50 microns to about 120 microns may be suitable for some embodiments of the first contact layer 405 and the second contact layer 410. The spacer layer 415 may be a flexible foam in some examples. The profile of the spacer layer 415 may also provide flexibility. In the example of FIG. 4, the spacer layer 415 has a star profile having a plurality of appendages, such as spacer legs 425, coupled to and radiating from a central body 430. The spacer legs 425 in such a configuration can be manipulated to conform to various types of tissues sites having complex geometries. Other suitable profiles may include interconnected concentric rings or arcs, or some combination of appendages, rings, and arcs, which may be coupled to or form a central body. In some examples, the spacer legs 425 or other appendages may comprise a plurality of joints 435, which can further increase flexibility.

FIG. 5 is a top view of the tissue interface 120 of FIG. 4, as assembled, illustrating additional details that may be associated with some examples. As illustrated in the example of FIG. 5, the first contact layer 405 (not visible) and the second contact layer 410 can be geometrically similar and may be congruent in some embodiments. A plurality of bonds may be used to couple the first contact layer 405 to the second contact layer 410. The bonds may be formed using any known technique, including without limitation, welding (e.g., ultrasonic or RF welding), bonding, adhesives, cements, or other bonding technique or apparatus. In the example of FIG. 5, the bonds include peripheral bonds 505, spacer bonds 510, and directional bonds 515.

The peripheral bonds 505 may be disposed around a periphery of the first contact layer 405 and the second contact layer 410. The spacer bonds 510 can be disposed around the spacer layer 415, which can secure the spacer layer 415 in a fixed position relative to the first contact layer 405 and the second contact layer 410. In some embodiments, the directional bonds 515 can define one or more flow paths 520 toward the central body 430. For example, the directional bonds 515 are disposed between the spacer legs 425, and generally extend radially between the central body 430 and the peripheral bonds.

FIG. 5 also illustrates a plurality of sensors 530 that may be associated with tissue interface 120 of FIG. 4. The sensors 530 are disposed at various points within tissue interface 120, such that when the tissue interface 120 is applied within the abdominal cavity of a patient, the sensors 530 are positioned in different regions, zones, or areas within the abdominal cavity. This enables the sensors to measure and report such physical parameters as temperature level, moisture level, and pH level, among other values, at the different regions. The sensors 530 may be capable of transmitting and receiving signals wirelessly, which can enable the sensors to be remotely monitored and controlled by an external device (e.g., iPad or similar tablet device) without the need for physical wires or other invasive equipment.

In the exemplary embodiment in FIG. 5, the sensors 530 are mounted on the spacer layer 415. The sensors 530 may be fixedly attached to the material of the spacer layer 415. Alternatively, the sensors 530 may be removably attached to the spacer layer 415, thereby enabling the positions of the sensors 530 to be customized by moving the sensors 530 to target specific zones in the abdominal cavity. In FIG. 5, the sensors 530 are mounted proximate the distal ends of the spacer legs 425 (or appendages 425). This is by way of example only and should not be construed to limit the scope of the disclosure. In alternate embodiments, one or more of the sensors 530 may be mounted closer to, or actually on, the central body 430 of spacer layer 415. In still other embodiments, the sensors 530 may not be mounted on spacer layer 415 at all but may instead, be fixedly or removably attached between the spacer legs 425, such as in one of more of the flow paths 520.

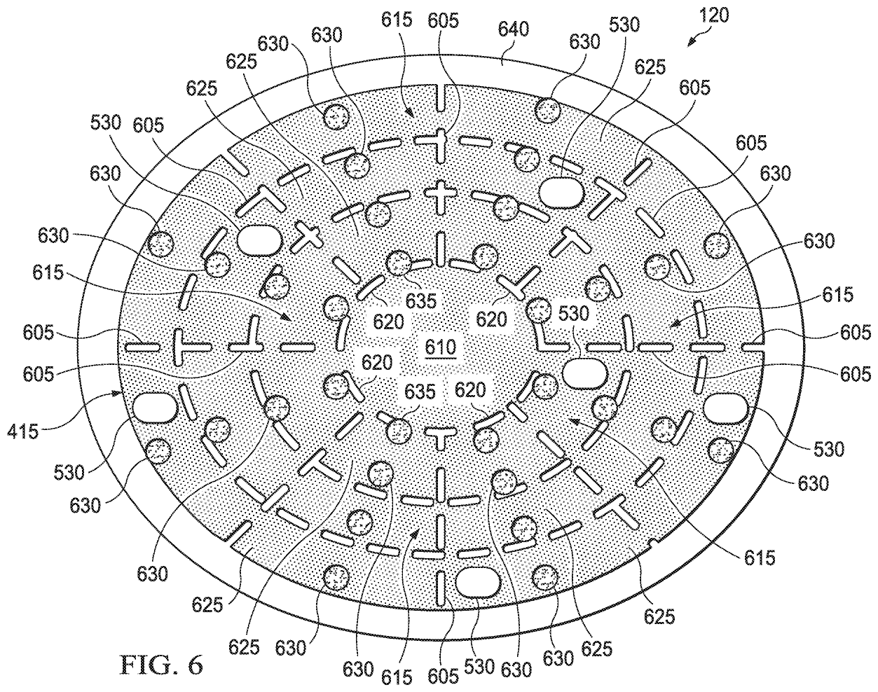

FIG. 6 is a top view of another example of the tissue interface 120, illustrating additional details that may be associated with some embodiments. FIG. 6 illustrates an exemplary pattern of perforations 605 that may be associated with some embodiments of the tissue interface 120. In the example of FIG. 6, a plurality of the perforations 620 demarcate a division between a central region 610 of the spacer layer 415 and a perimeter region 615. The plurality of perforations 620 can also be seen to define a plurality of border sub-regions 625 within the perimeter region 615. In some embodiments, the perimeter region 615 may include a plurality of bonds 630, as illustrated in the example of FIG. 6. The bonds 630 can couple a layer above the spacer layer 415 to a layer below the spacer layer 415. In some embodiments, one or more of the bonds 630 can be made by welding the layer above to the layer below through the spacer layer 415. The spacer layer 415 may have perforations corresponding to the bonds 630 to facilitate welding in some examples, but the layers may be welded through the manifold in other examples. In some embodiments, a portion of the welds 630 may include captivating bonds 635, which represent the innermost of the welds 630 within the perimeter region 615, closest to the central region 610. A further coupling of the layer above the spacer layer 415 and the layer below the spacer layer 415 in FIG. 6 is shown as a sealing portion 640 located near the outer edge of the spacer layer 415.

In some embodiments, the perimeter region 615 may be concentric with the central region 610. In some embodiments, the perimeter region 615 may have a central point, such as a center of mass, that is located within the central region 610. Whatever the relative shapes of inner and outer regions, a perimeter region surrounds an inner region in all directions outward, for example from a center point or from any point located within the inner region. In other words, there can be a 360-degree continuity between the central region 610 and the perimeter region 615. For example, FIG. 6 shows the spacer layer 415 to be an elliptical cylinder and the central region 610 to be a smaller central elliptical cylinder. In this example, the perimeter region 615, which is illustrated as an elliptical hollow cylinder, completely surrounds the central region 610 in the top-view of FIG. 6. This example may seem to indicate a concentric symmetry with respect to the breadth of the perimeter region 615 of the spacer layer 415. However, that may not necessarily be the case, as long as the perimeter region 615 exhibits some portion completely surrounding the central region 610.

The bonds 630 can function to hold the tissue interface 120 together, while still allowing the tissue interface 120 to be manually sized. In some embodiments, the central region 610 may be retained in place by the captivating bonds 635, and in some examples, captivating bonds may define a boundary between the central region 610 and the perimeter region 615. In the case of the bonds 630 and the captivating bonds 635, the arrangement of the plurality of the bonds 630 and the captivating bonds 635 throughout the perimeter region 615 may advantageously be dispersed to allow one or more border sub-regions 625 to be removed without significantly compromising the coupling of the tri-layer assembly.

For example, in FIG. 6, the bonds 630 and captivating bonds 635 may be arranged in each quadrant corresponding to the division of the perimeter region 615 into border sub-regions 625 by the perforations 620. By doing so, even excision of one or several of these bonds 630 along with a border sub-region 625 could allow the remaining bonds 630 and captivating bonds 635 to sufficiently anchor the layers above and below the spacer layer 415 through the remaining spacer layer 415. Although these bonds are exemplified in FIG. 6 to be as few as one bond 630 or captivating bond 635 per border sub-region 625, the pattern of the bonds 630 and the captivating bonds 635 in the perimeter region 615 could be any that provide adequate physical coupling of the tri-layer assembly. In addition, the plurality of perforations 620 may provide visual indicia for guiding an external user to more easily customize the spacer layer 415 to fit a given tissue site, but the external user need not make use of that guide nor necessarily seek to make manual sizing easier.

FIG. 6 also illustrates a plurality of sensors 530 that may be associated with tissue interface 120 of FIG. 6. The sensors 530 may be wireless-capable sensors in some examples. In the exemplary embodiment in FIG. 6, the sensors 530 are mounted at various spaced-apart points of the spacer layer 415. The sensors 530 may be fixedly attached to the material of the spacer layer. Alternatively, the sensors 530 may be removably attached to the spacer layer. This enables the positions of the sensors 530 to be customized by moving the sensors 530 to target specific zones in the abdominal cavity.

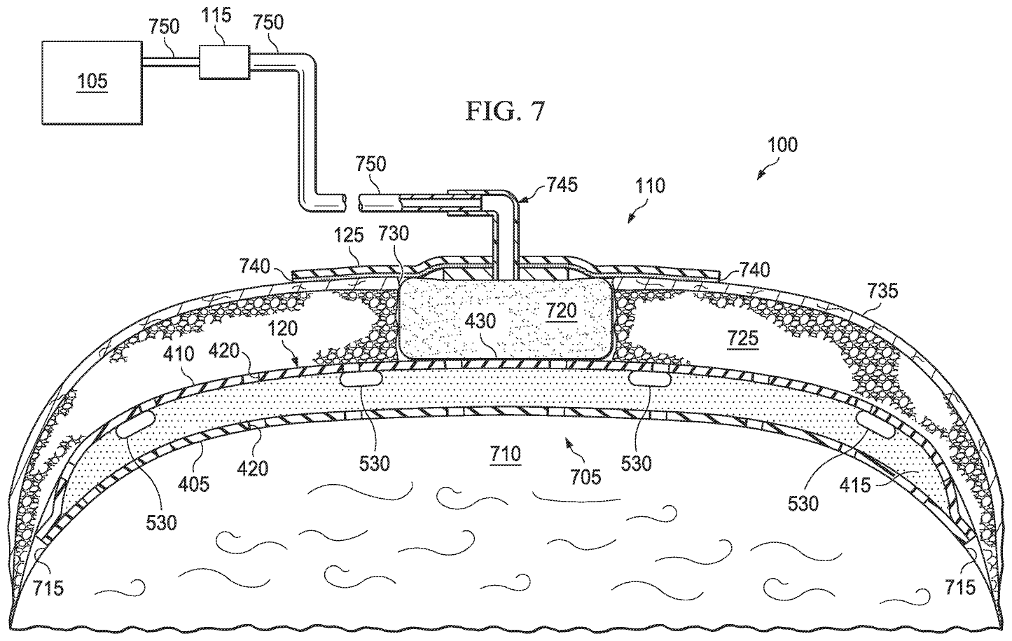

FIG. 7 is a schematic view of an example of the tissue interface 120 applied to a tissue site that comprises an abdominal cavity 705. The tissue interface 120 is flexible and can be inserted into the abdominal cavity 705. In the example of FIG. 7, the tissue interface 120 is supported by abdominal contents 710. A portion of the tissue interface 120, such as one or more of the spacer legs 425, may be disposed in or proximate to the paracolic gutter 715.

In the example of FIG. 7, the dressing 110 includes a filler manifold 720, which can be fluidly coupled to the tissue interface 120 and be configured to deliver negative pressure through the abdominal wall 725. For example, the filler manifold 720 may be inserted through an opening 730 in the abdominal wall 725 and disposed adjacent to the tissue interface 120 in fluid communication with at least some of the fenestrations 420 in the second contact layer 410. A plurality of sensors 530 are disposed on the surface of spacer layer 415, beneath second contact layer 410. The cover 125 may be placed over the opening 730 and sealed to epidermis 735 around the opening 730. For example, an attachment device such as an adhesive layer 740 may be disposed around a perimeter of the cover 125 to secure the cover 125 to the epidermis 735.

FIG. 7 further illustrates an example of a dressing interface 745 fluidly coupling the dressing 110 to a fluid conductor 750. The dressing interface 745 may be, as one example, a port or connector, which permits the passage of fluid from the filler manifold 720 to the fluid conductor 750 and vice versa. The dressing interface 745 of FIG. 7 comprises an elbow connector. Fluid collected from the abdominal cavity 705 may enter the fluid conductor 750 via the dressing interface 745. In other examples, the therapy system 100 may omit the dressing interface 745, and the fluid conductor 750 may be inserted directly through the cover 125 and into the filler manifold 720. In some examples, the fluid conductor 750 may have more than one lumen. For example, the fluid conductor 750 may have one lumen for negative pressure and liquid transport and one or more lumens for communicating pressure to a pressure sensor.

A negative pressure may be applied to the central body 430 or elsewhere to cause fluid flow through the fenestrations 420. The fenestrations 420 can allow fluid to be collected or distributed through and across the first contact layer 405 and the second contact layer 410 under negative pressure. Fluid can move directly or indirectly towards the negative-pressure source 105 through the fenestrations 420. In some examples, additional features such as the directional bonds 515 may direct flow toward the central body 430. For example, fluid can move through the spacer layer 415, through micro-channels formed between the first contact layer 405 and the second contact layer 410, or both. Negative pressure may be distributed more directly through the spacer layer 415, and can be the dominant pathway. In some examples, the spacer layer 415 may be omitted and fluid can move through micro-channels formed between the first contact layer 405 and the second contact layer 410.

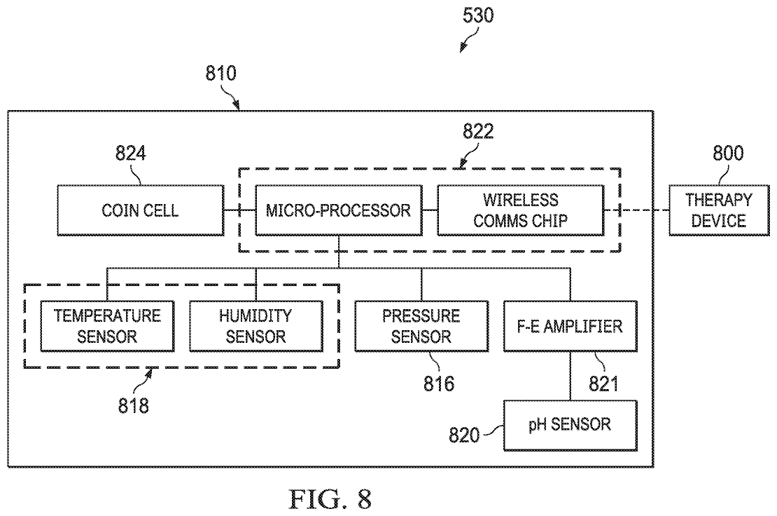

FIG. 8 is a schematic diagram of one or more of the sensors 530 associated with exemplary tissue interface 120 associated with exemplary tissue interface 120. The sensor 530 may comprise a housing 810 that contains a circuit board (not shown) on which are mounted a pressure sensor 816, a humidity sensor 818, a pH sensor 820, a front-end amplifier 821, a communications module 822, and a power source 824. The sensor 530 may comprise a temperature sensor that is a component of either the pressure sensor 816 or the humidity sensor 818. The sensor 530 is capable of two-way wireless communication with therapy device 800, which may be, for example, an iPad, a PC, or another similar device. The communications module 822 further comprises a controller (e.g., a microprocessor) and a wireless communication chip that communicates with the therapy device 800 under control of the microprocessor. The housing 810 provides a moisture-proof enclosure for the internal circuit board and the components mounted thereon. In some embodiments, the therapy device 800 may be a stand-alone device that simply monitors sensors 530 and displays values to an operator. In other embodiments, therapy device 800 may communicate with controller 130 to transmit the measured pressure, humidity, and pH to controller 130 in order to assist controller 130 in performing therapy. In still other embodiments, therapy device 800 may be an integral part of controller 130.

Using a wireless communications module 822 has the advantage of eliminating an electrical conductor between the tissue interface 120 and the therapy device 800 that may become entangled with the fluid conductor 650 when in use during therapy treatments. The wireless communication chip in wireless communications module 822 may comprise an integrated device that implements Bluetooth® Low Energy wireless technology. More specifically, the communications module 822 may be a Bluetooth® Low Energy system-on-chip that includes a microprocessor, such as the nRF51822 chip available from Nordic Semiconductor. The wireless communications module 822 may be implemented with other wireless technologies suitable for use in the medical environment.

In some embodiments, the power source 824 may be, for example, a battery that may be a coin cell battery having a low-profile that provides a 3-volt source for the communications module 822 and the other electronic components within sensor 530. In some example embodiments, all of the components within housing 810 associated with the sensor 530 may be integrated into a single package.

Each of the component sensors of sensor 530 may comprise a sensing portion (or probe) that extends outside of housing 810 in order to make contact with fluids in the tissue interface 120 so that temperature, pressure and pH may be measured. The front-end amplifier 821 amplifies the measured pH value detected by pH sensor 820.

In some embodiments, the pressure sensor 816 may be a piezo-resistive pressure sensor having a pressure sensing element covered by a dielectric gel such as, for example, a Model 1620 pressure sensor available from TE Connectivity. The dielectric gel provides electrical and fluid isolation from the bodily fluids in order to protect the sensing element from corrosion or other degradation.

In some examples, the pressure sensor 816 may comprise a temperature sensor for measuring the temperature of the fluids in the tissue interface 120. In other embodiments, the humidity sensor 818 may comprise a temperature sensor for measuring the temperature. In some embodiments, the humidity sensor 818 that also comprises a temperature sensor may be a single integrated device such as, for example, Model HTU28 humidity sensor also available from TE Connectivity.

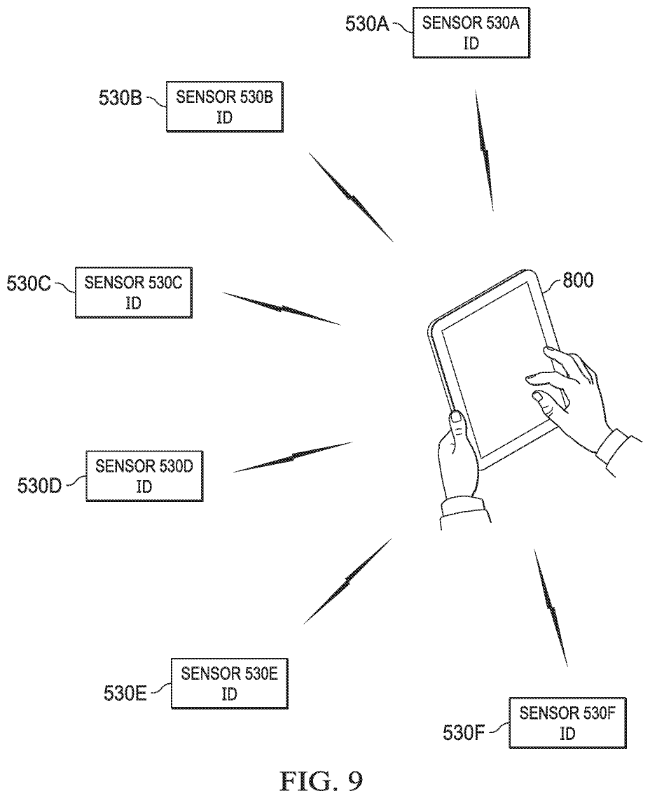

FIG. 9 is a network topology diagram illustrating the operation of a plurality of wireless sensors 530A-530F similar to the wireless capable sensor 530 in FIG. 8. Each of the sensors 530A-503F comprises a unique sensor identifier (ID) value that enables therapy device 800 to determine the identity of each sensor 530A-530F and to establish a dedicated bi-directional communication link to each sensor 530A-530F. For example, sensor 530A ID may be a unique binary value associated with sensor 530A, sensor 530B ID may be a unique binary value associated with sensor 530B, and so forth. In an exemplary embodiment, the sensor ID value may be an embedded serial number associated with the wireless communication chip in the communications modules 822 in sensors 530A-530F. Preferably, the sensor ID value is also printed on the exterior of the housing 810 of each sensor 530A-530F in hexadecimal format, decimal format, alphanumeric format, or the like.

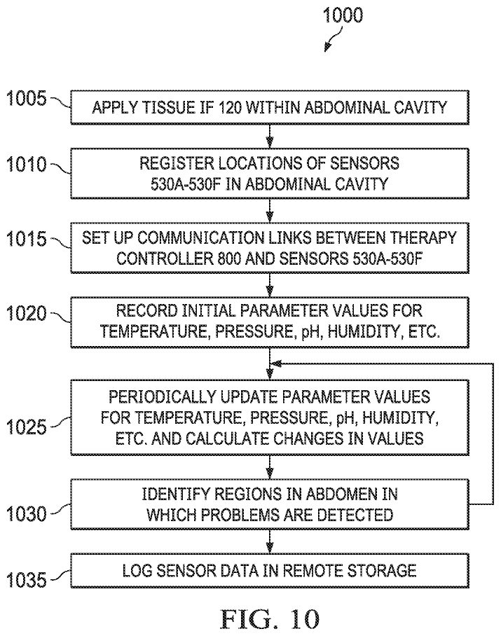

FIG. 10 is a flow diagram 1000 illustrating an example of operation of the wireless capable sensors in FIG. 8. Initially in 1005, the tissue interface 120 may be applied within abdominal cavity. Once the tissue interface 120 is in place, the operator registers in 1010 the locations or regions of the sensors 530A-530F in the abdominal cavity. In this manner, the parameter values registered by the sensors 530A-530F may be associated with specific regions within the abdominal cavity. Next in 1015, under operator control, the therapy device 800 sets up communication links between therapy controller 800 and each one of sensors 530A-530F.

At the start of a treatment in 1020, therapy device 800 transmits command messages to the sensors 530A-530F and records the initial parameter values for temperature, pressure, pH, humidity, and other parameters as may be desired in a particular application. Thereafter, therapy device 800 in 1025 may periodically (or aperiodically) transmits command messages to the sensors 530A-530F in order to update the parameter values for temperature, pressure, pH, humidity, and the like, and calculate changes in the measured parameter values for each sensor 530A-530F. Problems in the abdominal cavity may be detected by out-of-tolerance temperature values, pressure values, moisture (humidity) values, and pH values. Therapy device 800 in 1030 identifies regions in abdomen in which problems are detected by associating the out-of-tolerance values with the sensor ID value of each sensor 530A-530F that detects an out-of-tolerance value. Optionally, therapy device 800 in 1035 may log sensor data in remote storage.

Advantageously, the therapy system 100 utilizes the introduction and implementation of wireless sensors 530 to further automate and interconnect data collection and communication capabilities. In some exemplary embodiments, a system using negative-pressure abdominal therapy technology may include an integrated low-power electronics system of additional wireless sensors within its construction to further automate and interconnect its operation with that of a powered negative-pressure device and provide telemetry to provide data and status values such as: i) wound pressure, ii) fluid detection, iii) pH of liquid; iv) temperature; v) enhanced alarms, and vi) therapy duration and non-therapy time.

Wound pressure—In one variant, the therapy system may be configured to determine the pressure at multiple discrete points in the abdominal cavity by use of pressure sensors in sensor 530. The pressure feedback from the abdominal cavity may be wirelessly connected to a pressure regulator circuit in a control loop such that the system responds and reacts to pressure in the cavity rather than pressure at the container 115 or the negative-pressure source 105. As such, the sensor 530 may inform therapy device 800 (and the operator) of the current wound site pressure and may use a regulator monitoring approach to determine flow. By monitoring cavity pressure, the therapy device 800 is able to distinguish between a canister-full condition and other blockage conditions, such as a blockage of tubing.

Fluid detection—In one variant, the therapy system may be configured to detect the presence of liquid at various discrete points of the abdominal cavity by use of humidity and/or temperature sensors in sensor 530 disposed at discrete points in or on the dressing. This can be useful to understand if the fluid has been distributed evenly or if it is pooling in one area of the abdomen and may be particularly useful with therapy systems that provide controlled fluid instillation. In fluid instillation, it can be difficult to determine if the fluid has been distributed evenly, if it is stagnated, if it is pooling in one area of the abdomen, or if the presence of potential sepsis is detected. The change in humidity across the field may even be presented via a graphic on the therapy device 800, thus helping illustrate to the operator that the complete abdomen has been irrigated evenly during a controlled fluid instillation cycle.

Liquid pH—The pH of fluids at various discrete points of the abdominal cavity can be useful for understanding if the fluid is stagnated or if potential intra-abdominal sepsis is detected. This information can be used to detect the presence of bowel perforations or intra-abdominal sepsis and to improving overall abdominal wound management and healing.

Temperature—In one variant, the therapy system can be configured to determine the temperature at multiple discrete points in the abdominal cavity by use of remote temperature sensors in sensors 530. This can be particularly useful as multiple, discretely-located temperature sensors are able to form a de facto temperature gradient field identifying any higher temperatures or potential sources and locations of infection. This can even be presented via a graphic on the therapy device 800 as a field thus helping the operator locate the zone or area of potential intra-abdominal infection.

Enhanced alarms—Enhanced alarms may include, for example, a leak alarm, a blockage alarm, a canister-full alarm, and the like. The sensor 530 can provide feedback for abdominal pressure and therefore an ability to sense full canisters or blockages in individual arms or regions of tissue interface 120. The pressure sensors at various discrete points are able to detect large differences in pressure in each individual arm or region and therefore can detect a leak, blockage, or canister full event and also distinguish between them.

In an exemplary embodiment, the operator may obtain a standard disposable non-telemetry dressing system and add on packs of user-fit, customizable, single-use wireless monitor sensors 530, which provide the additional functionality described herein. In some embodiments, the single-use wireless sensor 530 modules may be pre-attached to the dressing and may have trimmable tubing or conduits that ensure measurements are recorded at a perimeter of the dressing.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 110, the container 115, or both may be eliminated or separated from other components for manufacture or sale. In other example configurations, the controller 130 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

What is claimed is:

1. A system for applying negative-pressure therapy to an abdominal cavity, the system comprising:
a tissue interface comprising a first contact layer and a second contact layer, each of the first contact layer and the second contact layer having perforations formed therein;
a spacer layer disposed between the first contact layer and the second contact layer, the spacer layer configured to extend to a plurality of different zones within the abdominal cavity;
a first sensor associated with the spacer layer and configured to acquire data associated with fluid at a first zone within the abdominal cavity; and
a first wireless transceiver associated with the first sensor and configured to transmit the data to an external therapy control device.

2. The system of claim 1, wherein the first sensor is configured to acquire data associated with the presence of fluid in the first zone.

3. The system of claim 1, wherein the first sensor is configured to acquire data associated with pressure in the first zone.

4. The system of claim 1, wherein the first sensor is configured to acquire data associated with temperature in the first zone.

5. The system of claim 1, wherein the first sensor is configured to acquire data associated with pH of fluid in the first zone.

6. The system of claim 1, wherein the spacer layer comprises an absorbent material.

7. The system of claim 1, further comprising:
a second sensor associated with the spacer layer and configured to acquire data associated with a second zone within the abdominal cavity; and
a second wireless transceiver associated with the second sensor and configured to transmit to the external therapy control device at least one data parameter associated with the second zone.

8. The system of claim 7, wherein the second sensor is configured to acquire data associated with a presence of fluid in the second zone.

9. The system of claim 8, wherein the second sensor is configured to acquire data associated with at least one of: i) a pressure of fluid in the second zone; ii) a temperature of fluid in the second zone; and iii) a pH of fluid in the second zone.

10. The system of claim 9, wherein the external therapy control device is configured to determine a difference between a first parameter value associated with fluid in the first zone and a corresponding first parameter value associated with fluid in the second zone.

11. A method for applying negative-pressure therapy to an abdominal cavity, the method comprising:
inserting in the abdominal cavity a tissue interface comprising:
a perforated first contact layer and a perforated second contact layer;
a spacer layer disposed between the perforated first and contact layers and configured to extend to a plurality of different zones within the abdominal cavity; and
a plurality of wireless-enabled sensors configured to acquire data associated with fluid at the plurality of different zones within the abdominal cavity;
from an external therapy control device, transmitting a first command message to a first wireless-enabled sensor configured to acquire data associated with fluid at a first zone within the abdominal cavity; and
transmitting the acquired data to the external therapy control device from the first wireless-enabled sensor.

12. The method of claim 11, wherein the first wireless-enabled sensor detects a presence of fluid in the first zone.

13. The method of claim 11, wherein the first wireless-enabled sensor detects a pressure of fluid in the first zone.

14. The method of claim 11, wherein the first wireless-enabled sensor detects a temperature of fluid in the first zone.

15. The method of claim 11, wherein the first wireless-enabled sensor detects a pH of fluid in the first zone.

16. The method of claim 11, wherein the spacer layer comprises an absorbent material.

17. The method of claim 11, further comprising:
from the external therapy control device, transmitting a second command message to a second wireless-enabled sensor configured to acquire data associated with fluid at a second zone within the abdominal cavity; and
transmitting the acquired data to the external therapy control device from the second wireless-enabled sensor.

18. The method of claim 17, wherein the second wireless-enabled sensor detects a presence of fluid in the second zone.

19. The method of claim 18, wherein the second wireless-enabled sensor detects at least one of: i) a pressure of fluid in the second zone; ii) a temperature of fluid in the second zone; and iii) a pH of fluid in the second zone.

20. The method of claim 19, wherein the external therapy control device is configured to determine a difference between a first parameter value associated with fluid in the first zone and a corresponding first parameter value associated with fluid in the second zone.
